(19) United States
(12) Patent Application Publication
(10) Pub. No.: US 2023/0133772 A1
(43) Pub. Date: May 4, 2023

(54) Cannabinoid Product for Improvement of Sleep

(71) Applicant: ECS Brands Ltd., Broomfield, CO (US)

(72) Inventor: Arthur Jaffee, Denver, CO (US)

(73) Assignee: ECS Brands Ltd, Broomfield, CO (US)

(21) Appl. No.: 17/946,635

(22) Filed: Sep. 16, 2022

Related U.S. Application Data
(63) Continuation-in-part of application No. 17/514,387, filed on Oct. 29, 2021.

(51) Int. Cl. A61K 31/352; A61K 31/05; A61K 36/185

(52) U.S. Cl. CPC A61K 31/352; A61K 31/05; A61K 36/185

(57) ABSTRACT

Cannabinoids are increasingly found to have beneficial effects on the human body, even without the intoxicating effects from THC. Herein we describe a cannabinoid which is used to assist with improvement of sleep. Tetrahydrocannabivarin, typically but not necessarily combined with assorted cannabinoids, provides a product for improvement of sleep without the need for diet, exercise, or other pharmaceuticals.

### CROSS REFERENCE TO RELATED APPLICATION

This application is a continuation-in-part (CIP) of U.S. Ser. No. 17/514,387 filed Oct. 29, 2021 and titled “Cannabinoid Product for Assistance with Weight Loss”.

### BACKGROUND

### Field of the Invention

The present invention relates generally to cannabinoids which assist with improvement of sleep. More specifically, the present invention relates to varin-rich cannabinoid extracts, such as tetrahydrocannabivarin, and its associated consumption for assist with sleep.

### Description of the Related Art

Good quality sleep is a critical component of a healthy lifestyle. Studies have shown that getting less sleep than normal tends to leave individuals tired, irritable, less creative, less emotionally receptive, and less open to new ideas. Subjects suffering from insomnia, diagnosed or otherwise, are at risk for poor physical and mental performance during waking hours. In combination with an unhealthy lifestyle, poor sleep can result in declines in the workplace, declines in the familial space, and general malaise that is difficult to pinpoint and treat.

A 2009 study of sleep disturbance in obese populations reported that 84.8% of obese patients reported at least one sleep disturbance symptom. In addition, men and women were shown to exhibit different symptoms. Women were found to be affected most by daytime sleepiness, night sweats, difficulties falling asleep, and difficulties staying asleep. Men were affected most by fatigue, night sweats, and difficulty falling asleep.

A variety of benefits exist from getting proper amounts and quality of sleep. Chronic sleep debt may lead to the development of mood disorders like depression and anxiety. Lack of sleep can lead to type II diabetes, primarily related to the handling of blood glucose. Long standing sleep deprivation was also associated with increased heart rate, blood pressure, and inflammation markers, leading to increased incidence of heart disease. In general, sleep plays a crucial role in healthy regulation of biological processes, and disturbance of that regulation may result in declines in health.

The most common suggestions related to improvement in sleep are pharmaceuticals, improving sleep hygiene, improving diet, and increasing exercise. While improving sleep with the use of pharmaceuticals is the most directly effective, it is not without risks.

Of the many pharmaceuticals are taken to assist with sleep, the most frequently taken are hypnotics. Hypnotics run a number of risks, and even negative side effects related to sleep. While hypnotics improve the ability to fall asleep quickly, a number interfere with REM sleep. Many hypnotics have high risk of overdose or substance dependence with prolonged use, such as barbiturates and benzodiazepines. Other pharmaceuticals taken to assist with sleep are used off-label, and have limited clinical effectiveness, such as melatonin and antihistamines.

Sleep hygiene is a behavioral and environmental practice to assist with quality of sleep. The primary recommendations are to establish a sleep schedule, reducing naps, limiting activities prior to bed, reducing consumption of alcohol and stimulants in the hours prior to bedtime, and having a peaceful, comfortable, and dark sleep environment. While this practice has been commonplace since the 1970s, recent studies have shown that the effectiveness of sleep hygiene is limited for the general population.

Studies are showing that diet and nutrition are affecting sleep quality. Studies have shown that a diet high in carbohydrates promoted reduced time of wakefulness in bed prior to sleep as compared to a diet high in fat, mixed micronutrients and macronutrients are needed to promote quality sleep, varied diet of fresh fruits and vegetables, whole grains, and low in saturated fat may be optimal for improving sleep quality. However, there are no existing clinical studies on long-term dietary practices as they relate to sleep quality.

Diet regimes, require substantial commitment to a restrictive diet, and to maintain the commitment for an extended period of time. The early stages of diets can be particularly challenging for those undertaking them as the restrictive nature of the diet requires additional time commitments for determining which, and how much, foods should be consumed. Additionally, the “on diet” meals may be not to the palette of those undertaking the diet as the flavor profile is likely very different from the flavor profile of the typical meal consumed when not on the diet. While dieting, social events also present frequent, and occasionally awkward, challenges as it is frequent that dieting at a social event will be difficult at best. This results in convenient opportunities for “cheat days” which undermine the results of the diet.

How exercise and physical activity improves sleep is currently unknown in medical science. However, it is known that moderate aerobic exercise increased the amount of deep sleep achieved. Exercising too late, though, can have a negative impact on sleep. Aerobic exercise releases endorphins which keep you awake. Additionally, exercising raises core body temperature, which also signals to the body that it is time to be awake.

Furthermore, exercise requires substantial commitment in both financial and temporal terms. These are frequent barriers. Financial investments range from one-time costs such as home equipment to recurring costs such as gym memberships and things such as new athletic shoes. The temporal investment required to maintain the commitment to exercise alone is substantial and well-studied outside of this patent application. The personal discipline required to maintain the financial and temporal commitment to an exercise regimen, in the face of muscular soreness and overall fatigue, is another barrier to individuals wishing to improve sleep quality through exercise.

### SUMMARY

In accordance with the embodiments herein, a cannabinoid product to be consumed to assist with sleep. The cannabinoid product described herein contains tetrahydrocannabivarin. Other cannabinoids may optionally be included as part of the product. The other cannabinoids are typically cannabidiol, cannabidivarin, cannabigerol, cannabidiolic acid, cannabigerolic acid, or cannabinol.

### DETAILED DESCRIPTION OF EMBODIMENTS

In the following description, for purposes of explanation and not limitation, details and descriptions are set forth in order to provide a thorough understanding of the present invention. However, it will be apparent to those skilled in the art that the present invention may be practiced in other embodiments that depart from these details and descriptions without departing from the spirit and scope of the invention.

In an illustrative embodiment of the invention, the cannabinoid product is either naturally occurring or synthetic tetrahydrocannabivarin (“THCV”) consumed to assist with sleep in the person consuming the THCV. In optional embodiments, the THCV may be combined with at least one naturally occurring or synthetic assorted cannabinoids such as cannabidiol (“CBD”), cannabidivarin (“CBDV”), cannabigerol (“CBG”), cannabidiolic acid (“CBDA”), cannabigerolic acid (“CBGA”), and/or cannabinol (“CBN”). Other cannabinoids are contemplated.

In a further embodiment of the invention containing at least one assorted cannabinoid the cannabinoid product is between 1.0 and 95.0 percent by mass of THCV and between 5.0 and 99.0 percent by mass of the at least one assorted cannabinoid. In other embodiments with the assorted cannabinoids, the THCV is typically between 1.0 and 70.0 percent by mass, the CBD is between 25.0 and 50.0 percent by mass, and the remaining assorted cannabinoids are each between 5.0 and 35.0 percent by mass.

Frequently, the invention is within a carrier oil. This carrier oil is typically a hemp oil, such as golden hemp seed oil. Occasionally, the invention would be an emulsion within the carrier oil, however this is not necessary. In an emulsion, water solubility and smaller particle sizes are more ideal for absorption of the invention into the human body. For the invention as an emulsion within the carrier oil, particle sizes between 10.0 nm and 5.0 μm are preferred but not necessary. Larger and smaller particle sizes are contemplated and still allow the invention to function.

Multiple types of delivery systems can be used with the invention. Some embodiments are as an oil, while others are tinctures, gel capsules, liquid emulsions, powders, transdermal preparations, or even as an inhalant. Other types of delivery methods are contemplated.

Most typically, an individual will consume the invention to assist with sleep. The use of the invention as a sleep aid supplement has definitive advantages, namely that consumers are not expected to make major changes to their lifestyle or dietary habits when taking the invention. Individuals taking the invention for at least 90 days reported improvement in sleep quality without dietary restrictions or exercise regimens. By promoting natural, healthy improvement of sleep quality, it is expected that participants will be able to integrate themselves into healthier habits without the traditional rebound effects seen in many diet or exercise programs

Individuals taking the invention for at least 90 days with no other direct change to lifestyle, including diet and exercise, achieved reduction in the time spent awake in bed by at least 20.0 minutes, reduction in the time spent in REM sleep by at least 8.0 minutes, and improvement in the time spent in deep sleep by at least 70.0 minutes.

In another illustrative embodiment, the invention is a cannabinoid product containing hemp seed oil and a mixture of cannabinoids made up of 74.0% CBD, 17.0% CBDV, 5.0% THCV, 2.0% CBG, 1.0% CBDA, 1.0% CBGA, and less than 0.1% CBN, with the percentages of each cannabinoid being percent by mass. This embodiment may be taken to assist with sleep. It may be an emulsion; it may be an oil, tincture, gel capsule, liquid emulsion, powder, transdermal preparation, or even as an inhalant. Other forms of manufacture and delivery are contemplated.

What is claimed is:

1. A product, comprising:
tetrahydrocannabivarin; and
wherein the tetrahydrocannabivarin is used to assist with sleep in an individual consuming the tetrahydrocannabivarin.

2. The product of claim 1, further comprising:
at least one assorted cannabinoid, wherein the at least one assorted cannabinoid is selected from the group consisting of cannabidivarin, cannabigerol, cannabidiolic acid, cannabigerolic acid, cannabinol, and cannabidiol.

3. The product of claim 2, wherein
the tetrahydrocannabivarin is between 1.0 and 70.0 percent by mass;
the at least one assorted cannabinoid is cannabidiol and at least one additional assorted cannabinoid selected from the same grouping; and
the cannabidiol is between 25.0 and 50.0 percent by mass; and
the at least one additional assorted cannabinoid is between 5.0 and 35.0 percent by mass.

4. The product of claim 2, wherein;
the tetrahydrocannabivarin is between 1.0 and 95.0 percent by mass; and
the at least one assorted cannabinoid is cannabidiol and is between 5.0 and 99.0 percent by mass.

5. The cannabinoid product of claim 1, further comprising a carrier hemp oil.

6. The cannabinoid product of claim 1, wherein the cannabinoid product is prepared in a manner selected from the group consisting of oil, tincture, gel capsule, liquid emulsion, powder, transdermal preparation, and inhalant.

7. The cannabinoid product of claim 1, wherein the cannabinoid product is an emulsion.

8. The cannabinoid product of claim 7, wherein the emulsion has a particle size between 10.0 nm and 5.0 μm.

9. The cannabinoid product of claim 1, wherein the individual has reduced time awake in bed by at least 20.0 minutes after taking the cannabinoid product for at least 90 days.

10. The cannabinoid product of claim 1, wherein the individual has reduced time spent in REM sleep by at least 8.0 minutes after taking the cannabinoid product for at least 90 days.

11. The cannabinoid product of claim 1, wherein the individual has improved time spent in deep sleep by at least 70.0 minutes after taking the cannabinoid product for at least 90 days.

12. A cannabinoid product, comprising:
hemp seed oil; and
a cannabinoid mixture;
wherein the cannabinoid mixture comprises:
cannabidiol at 74.0 percent by mass;
cannabidivarin at 17.0 percent by mass;
tetrahydrocannabivarin at 5.0 percent by mass;
cannabigerol at 2.0 percent by mass;
cannabidiolic acid at 1.0 percent by mass;
cannabigerolic acid at 1.0 percent by mass; and
cannabinol at less than 0.1 percent by mass.

13. The cannabinoid product of claim 12, wherein an individual consumes the cannabinoid product to assist with sleep.

14. The cannabinoid product of claim 13, wherein the individual has reduced time awake in bed by at least 20.0 minutes after taking the cannabinoid product for at least 90 days.

15. The cannabinoid product of claim 13, wherein the individual has reduced time spent in REM sleep by at least 8.0 minutes after taking the cannabinoid product for at least 90 days.

16. The cannabinoid product of claim 13, wherein the individual has improved time spent in deep sleep by at least 70.0 minutes after taking the cannabinoid product for at least 90 days.

17. The cannabinoid product of claim 12, wherein the cannabinoid product is prepared in a manner selected from the group consisting of oil, tincture, gel capsule, liquid emulsion, powder, transdermal preparation, and inhalant.

18. The cannabinoid product of claim 12, wherein the cannabinoid product is an emulsion.

19. The cannabinoid product of claim 18, wherein the emulsion has a particle size between 10.0 nm and 5.0 μm.
